(19) United States
(12) Patent Application Publication
(10) Pub. No.: US 2025/0282893 A1
(43) Pub. Date: Sep. 11, 2025

(54) PREVENTION OF DIABETES BY ENHANCEMENT OF IMMUNE MODULATION

(71) Applicant: CREATIVE MEDICAL TECHNOLOGIES, INC., Phoenix, AZ (US)

(72) Inventors: Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 19/045,460

(22) Filed: Feb. 4, 2025

Related U.S. Application Data
(60) Provisional application No. 63/561,681, filed on Mar. 5, 2024.

(51) Int. Cl. C07K 16/42; A61K 9/00; A61K 35/28; A61K 35/50; A61K 35/51; A61K 45/06; A61P 3/10; A61P 37/04; G01N 33/68

(52) U.S. Cl. CPC C07K 16/4241; A61K 9/0019; A61K 35/28; A61K 35/50; A61K 35/51; A61K 45/06; A61P 3/10; A61P 37/04; G01N 33/6854; G01N 33/6893; G01N 2333/46; G01N 2333/805; G01N 2800/52

(57) ABSTRACT

Methods of reducing death, destruction or rejection of beta islets along with peripheral receptors in patients before they have diabetes, wherein said method comprising the steps of: a) obtaining a patient in need of immune cell therapy; b) administering to said patient one or more immunomodulatory cells; c) assessing the prevention and transition to diabetes.

### CROSS-REFERENCE TO RELATED APPLICATIONS

The present application claims benefit of U.S. Provisional Patent Application Ser. No. 63/561,681, filed on Mar. 5, 2024 entitled PREVENTION OF DIABETES BY ENHANCEMENT OF IMMUNE MODULATION, the contents of which are incorporated herein by reference in its entirety.

### FIELD OF THE INVENTION

The invention pertains to the field of diabetes. More specifically, the invention pertains to prevention of diabetes through the administration of enhanced immunomodulatory cells from allogeneic, autologous, or xenogeneic sources individually or in combination. The invention pertains to the field of increasing beta islet cell survival and reduction/prevention of immunological destruction of said cell and associated cells types. It also promotes beta islet cell regeneration, suppress inflammation, decrease islet beta-cell autoimmunity and enhance regulatory T-cell functions. It also pertains to the increased sensitivity of insulin receptors found on muscle bodies.

### BACKGROUND OF THE INVENTION

Diabetes stands as a significant contributor to both illness and death worldwide. Statistics indicate that approximately one in every seven healthcare dollars is allocated to managing diabetes and its associated complications. There exist two primary variations of diabetes: Type 1 and Type 2. Type 1 diabetes, also recognized as insulin-dependent diabetes mellitus (IDDM) or juvenile diabetes, involves minimal to no insulin production by the pancreas, often stemming from autoimmune attacks on pancreatic beta-cells. It constitutes one of the most financially burdensome chronic childhood diseases, persisting into adulthood. Estimates suggest over one million Americans live with IDDM. Individuals with full-blown IDDM necessitate multiple daily insulin injections or continuous insulin infusion via a pump, alongside frequent blood sugar monitoring, typically involving finger-prick tests six or more times a day. Neither dietary adjustments nor oral hypoglycemic agents suffice; only insulin treatment proves effective. Without intervention, ketonemia and acidosis, stemming from insulin secretion loss, may escalate to diabetic coma. Despite meticulous insulin administration, various factors—such as stress, hormones, growth, physical activity, medications, illness, or fatigue—impair insulin utilization, leading to multiple complications. Despite its lower prevalence compared to type 2 diabetes—constituting only 10-20% of cases—type 1 diabetes exerts a substantial toll on morbidity and mortality due to its onset occurring at a younger age. The primary cause of type 1 diabetes lies in the autoimmune assault on pancreatic islet beta cells. This attack triggers both cellular and humoral immune responses targeting various beta cell autoantigens, often preceding clinical symptoms. Notably, the presence of antibodies against specific autoantigens such as glutamic acid decarboxylase (GADA), insulinoma-associated antigen (1A-2A), or insulin (IAA), alone or in combination, serves as a predictive marker for type 1 diabetes. At the time of diagnosis, approximately 80-90% of type 1 diabetes patients exhibit islet-cell antibodies (ICA), 1A-2A, and GADA. Furthermore, these autoantibodies, particularly GADA, may also manifest in approximately 10% of initially classified type 2 diabetes cases among adults, a condition termed Latent Autoimmune Diabetes in Adults (LADA). Although LADA shares certain genetic susceptibility factors and type 1 diabetes-associated autoantibodies with type 1 diabetes, its progression towards insulin dependence tends to occur at a slower pace.

On the other hand, Non-Insulin Dependent Diabetes Mellitus (NIDDM), also termed adult-onset diabetes, associates with peripheral tissue insulin response impairment. Roughly 18.2 million Americans endure NIDDM, with a concerning trend of diagnosis in younger individuals due to the obesity epidemic. The economic repercussions of NIDDM are evident, given the high healthcare costs involved. Insulin resistance characterizes most obese individuals; initially, compensatory beta-cell insulin production counters hyperglycemia. However, protracted insulin resistance diminishes beta-cell capacity, culminating in postprandial hyperglycemia. This transition precipitates hepatic gluconeogenesis-induced fasting hyperglycemia. Unlike IDDM, NIDDM manifests minimal ketonemia and acidosis, often not necessitating insulin therapy. Long-term complication prevention remains the primary challenge, encompassing vascular, ocular, and renal systems. Though various agents enhance glucose sensitivity or insulin secretion, they fail to precisely mimic physiological insulin control. Consequently, glucose fluctuations lead to macrovascular complications like coronary atherosclerosis and microvascular issues such as macular degeneration and renal failure. Neuropathies frequently coexist with hyperglycemia. Treatment approaches for NIDDM vary based on individual characteristics and disease severity, with the overarching aim of normalizing plasma glucose levels. Numerous medical tests, including those monitoring glucose, cholesterol, and lipid levels, aid in disease management. Maintaining normal glucose levels serves as a key metric for preventing secondary complications like retinopathy, neuropathy, vascular disease, and strokes.

As there are limited options once onset of diabetes, cell therapy has been utilized to help improve glycemic control. However, to date no one has mechanistic analyzed how to modulate the immune system to prevent the antibody and t cell-based destruction of beta islets, their associated cells and receptors.

### SUMMARY OF THE INVENTION

Preferred embodiments are drawn to methods of reducing death, destruction or rejection of beta islets along with peripheral receptors in patients before they have diabetes, wherein said method comprising the steps of: a) obtaining a patient in need of immune cell therapy; b) administering to said patient one or more immunomodulatory cells; c) assessing the prevention and transition to diabetes

A method for preventing autoimmune type 1 and type 2 diabetes in a patient identified as having antibodies Islet Cell Antibodies (ICA): These antibodies target various proteins found in the islet cells of the pancreas, including insulin, glutamic acid decarboxylase (GAD), insulinoma-associated antigen-2 (IA-2), and zinc transporter 8 (ZnT8). Islet cell antibodies are present in the majority of individuals with newly diagnosed T1D. Glutamic Acid Decarboxylase Antibodies (GADA): GAD is an enzyme involved in the production of the neurotransmitter gamma-aminobutyric acid (GABA). Antibodies against GAD (GADA) are commonly found in individuals with T1D. They are among the earliest markers to appear in the autoimmune process leading to T1D. Insulin Autoantibodies (IAA): These antibodies target insulin itself. They are often detected in individuals who develop T1D, particularly in children. Insulinoma-Associated Antigen-2 Antibodies (IA-2A): IA-2 is a protein found in pancreatic beta cells. Antibodies against IA-2 (IA-2A) are associated with T1D and can be detected in individuals with the disease. Zinc Transporter 8 Antibodies (ZnT8A): Zinc transporter 8 is involved in the storage and secretion of insulin in pancreatic beta cells. Antibodies against ZnT8 (ZnT8A) are another marker associated with T1D and can be detected in individuals with the disease.

A summary of the invention is provided below with respect to the following numbered aspects.

1) A method for preventing autoimmune type 1 and type 2 diabetes in a patient identified as having antibodies associated with type 1 diabetes, comprising administering perinatal cells and/or T regulatory cells (Tregs) individually or in combination to said patient.

2) The method of claim 1, wherein said perinatal cells are obtained from umbilical cord blood/tissue.

3) The method of claim 1, wherein said perinatal cells are obtained from placental tissue.

4) The method of claim 1, wherein said perinatal cells are obtained from amniotic fluid.

5) The method of claim 1, wherein said perinatal cells are obtained from chorionic villi.

6) The method of claim 1, wherein said perinatal cells are administered via intravenous injection.

7) The method of claim 1, wherein said perinatal cells are administered via local injection via arterial supply or venous exit into pancreatic tissue.

8) The method of claim 1, wherein said perinatal cells are administered via subcutaneous/intramuscular, intraperitoneal depot/patch.

9) The method of claim 1, wherein said perinatal cells are administered via oral-endoscopic retrograde administration.

10) The method of claim 1, wherein said perinatal cells are administered via intranasal administration.

11) The method of claim 1, wherein said perinatal cells are administered in combination with Tregs.

12) The method of claim 11, wherein said Tregs are administered before, simultaneously with, or after the administration of perinatal cells before or after teplizumab or rhGAD65.

13) The method of claim 1, further comprising culturing said perinatal cells in vitro prior to administration to enhance therapeutic efficacy.

14) The method of claim 1, further comprising genetically modifying said perinatal cells to enhance therapeutic efficacy.

15) The method of claim 1, further comprising administering immunomodulatory agents to said patient to prevent rejection of said perinatal cells and Tregs.

16) The method of claim 1, further comprising administering growth factors to said patient to enhance the survival and function of said perinatal cells and Tregs.

17) The method of claim 1, further comprising encapsulating or protecting said perinatal cells and Tregs to prolong their survival and function in vivo.

18) A pharmaceutical composition for preventing type 1 and type 2 diabetes in a patient identified as having antibodies associated with type 1 diabetes, comprising perinatal cells, Tregs, and a pharmaceutically acceptable carrier or excipient.

19) The pharmaceutical composition of claim 18, further comprising immunomodulatory agents to prevent rejection of said perinatal cells and Tregs.

20) The pharmaceutical composition of claim 18, further comprising growth factors to enhance the survival and function of said perinatal cells and Tregs.

21) The pharmaceutical composition of claim 18, wherein said perinatal cells are genetically modified to enhance therapeutic efficacy.

22 The pharmaceutical composition of claim 18, wherein said perinatal cells and Tregs are encapsulated or protected to prolong their survival and function in vivo.

23) A kit for preventing type 1 and type 2 diabetes in a patient identified as having antibodies associated with type 1 diabetes, comprising a container containing perinatal cells, a container containing Tregs, and instructions for administering said perinatal cells and Tregs to said patient.

24) The kit of claim 23, further comprising immunomodulatory agents and/or growth factors, including teplizumab or rhGAD65.

25) The kit of claim 23, further comprising encapsulation materials for protecting said perinatal cells and Tregs from immune rejection or destruction.

26) A method for isolating and expanding perinatal cells for use in preventing type 1 and type 2 diabetes in a patient identified as having antibodies associated with type 1 diabetes, comprising: isolating perinatal cells from a perinatal tissue source; culturing said perinatal cells in vitro; expanding said perinatal cells to obtain a therapeutically effective number of cells; and administering said perinatal cells to said patient.

27) The method of claim 26, wherein said perinatal tissue source is umbilical cord tissue/blood.

28) The method of claim 26, wherein said perinatal tissue source is placental tissue.

29) The method of claim 26, wherein said perinatal tissue source is amniotic fluid.

30) The method of claim 26, wherein said perinatal tissue source is chorionic villi.

31) The method of claim 26, further comprising genetically modifying said perinatal cells to enhance therapeutic efficacy.

32) The method of claim 26, further comprising encapsulating or protecting said perinatal cells to prolong their survival and function in vivo.

33) A method for isolating and expanding Tregs for use in preventing type 1 and type 2 diabetes in a patient identified as having antibodies associated with type 1 diabetes, comprising: isolating Tregs from a tissue source; culturing said Tregs in vitro; expanding said Tregs to obtain a therapeutically effective number of cells; and administering said Tregs to said patient.

34) The method of claim 33, wherein said tissue source is peripheral blood.

35) The method of claim 33, wherein said tissue source is lymph nodes.

36) The method of claim 33, further comprising genetically modifying said Tregs to enhance therapeutic efficacy.

37) The method of claim 33, further comprising encapsulating or protecting said Tregs to prolong their survival and function in vivo via enhanced culture conditions.

38) A method for preventing recurrence of type 1 and type 2 diabetes in a patient identified as having antibodies associated with type 1 diabetes, comprising administering booster doses of perinatal cells and/or Tregs to said patient at predetermined intervals.

39) The method of claim 38, wherein said booster doses are administered annually.

40) The method of claim 38, wherein said booster doses are administered biannually.

41) The method of claim 38, wherein said booster doses are administered quarterly.

42) The method of claim 38, wherein said booster doses are administered monthly.

43) A method for monitoring the efficacy of perinatal cell and/or Treg therapy for preventing type 1 and type 2 diabetes in a patient identified as having antibodies associated with type 1 diabetes, comprising: measuring levels of pancreatic islet cell autoantibodies in the patient; monitoring blood glucose levels in the patient; and assessing insulin production and function in the patient.

44) The method of claim 43, wherein a decrease in pancreatic islet cell autoantibody levels, stabilization of blood glucose levels, and restoration of insulin production and function indicate efficacy of perinatal cell and Treg therapy.

45) The method of claim 43, further comprising imaging pancreatic tissue to assess regeneration and preservation of pancreatic islet cells.

46) The method of claim 43, wherein said monitoring is performed periodically following administration of perinatal cells and Tregs.

47 The method of claim 43, wherein said monitoring is performed using non-invasive imaging techniques.

48) The method of claim 43, wherein said monitoring is performed using biomarker assays.

49) The method of claim 43, wherein said monitoring is performed using clinical assessments.

50) A method for selecting patients for perinatal cell and Treg therapy for preventing type 1 and type 2 diabetes, comprising: identifying patients with antibodies associated with type 1 diabetes; assessing pancreatic beta-cell function in said patients; and selecting patients with preserved pancreatic beta-cell function for perinatal cell and Treg therapy.

51) The method of claim 50, wherein said pancreatic beta-cell function is assessed using glucose tolerance tests.

52) The method of claim 50, wherein said pancreatic beta-cell function is assessed using C-peptide levels.

53) The method of claim 50, wherein said pancreatic beta-cell function is assessed using insulin secretion assays.

54) A method for preventing type 1 and type 2 diabetes in a patient identified as having antibodies associated with type 1 diabetes, comprising administering perinatal cells, Tregs, and a pharmaceutical composition comprising an immunomodulatory agent to said patient.

55) The method of claim 54, wherein said immunomodulatory agent is administered before, simultaneously with, or after the administration of perinatal cells and Tregs.

56) The method of claim 54, wherein said immunomodulatory agent is administered periodically to prevent destruction of said perinatal cells, beta islet, gamma, delta cells and/or Tregs.

57) The method of claim 54, wherein said immunomodulatory agent is administered in combination with growth factors to enhance the survival and function of said perinatal cells, beta islet, gamma, delta cells and/or Tregs.

58) The method of claim 54, wherein said perinatal cells, Tregs, and immunomodulatory agent are administered via different routes of administration.

59) A method for preventing type 1 and type 2 diabetes in a patient identified as having antibodies associated with type 1 diabetes, comprising administering perinatal cells, Tregs, and a pharmaceutical composition comprising a growth factor to said patient.

60) The method of claim 59, wherein said growth factor is administered before, simultaneously with, or after the administration of perinatal cells and/or Tregs.

61) The method of claim 59, wherein said growth factor is administered periodically to enhance the survival and function of said perinatal cells and/or Tregs.

62) The method of claim 59, wherein said perinatal cells, Tregs, and growth factor are administered via different routes of administration.

63) A method for preventing type 1 and type 2 diabetes in a patient identified as having antibodies associated with type 1 diabetes, comprising administering perinatal cells, Tregs, and a pharmaceutical composition comprising an encapsulating material to said patient.

64) The method of claim 63, wherein said encapsulating material is administered before, simultaneously with, or after the administration of perinatal cells and Tregs.

65) The method of claim 63, wherein said encapsulating material is administered periodically to prolong the survival and function of said perinatal cells and Tregs.

66) The method of claim 63, wherein said perinatal cells, Tregs, and encapsulating material are administered via different routes of administration.

67) A pharmaceutical composition for preventing type 1 and type 2 diabetes in a patient identified as having antibodies associated with type 1 diabetes, comprising perinatal cells, Tregs, and a pharmaceutically acceptable carrier, excipient or platelet derivative.

68) The pharmaceutical composition of claim 67, further comprising an immunomodulatory agent to prevent destruction of said perinatal cells and Tregs.

69) The pharmaceutical composition of claim 67, further comprising a growth factor to enhance the survival and function of said perinatal cells and Tregs.

70 The pharmaceutical composition of claim 67, further comprising an encapsulating material to prolong the survival and function of said perinatal cells and Tregs.

71) The pharmaceutical composition of claim 67, wherein said perinatal cells are genetically modified to enhance therapeutic efficacy.

72) The pharmaceutical composition of claim 67, wherein said perinatal cells and/or Tregs are encapsulated or protected to prolong their survival and function in vivo.

73) The pharmaceutical composition of claim 67, wherein said perinatal cells and/or Tregs are administered via different routes of administration.

74) A kit for preventing type 1 and type 2 diabetes in a patient identified as having antibodies associated with type 1 diabetes, comprising a container containing perinatal cells, a container containing Tregs, and a pharmaceutical composition comprising an immunomodulatory agent, a growth factor, a carrier solution or an encapsulating material, and instructions for administering said perinatal cells, Tregs, and pharmaceutical composition to said patient.

75) The kit of claim 74, wherein said perinatal cells and Tregs are administered via different routes of administration.

76) The kit of claim 74, further comprising growth factors to enhance the survival and function of said perinatal cells and Tregs.

77) The kit of claim 74, further comprising encapsulation materials for protecting said perinatal cells and Tregs from immune rejection or destruction.

78) A method for preventing recurrence of type 1 and type 2 diabetes in a patient identified as having antibodies associated with type 1 diabetes, comprising administering booster doses of perinatal cells, Tregs, and a pharmaceutical composition comprising an immunomodulatory agent, a growth factor, or an encapsulating material to said patient at predetermined intervals.

79) The method of claim 78, wherein said booster doses are administered annually.

80) The method of claim 78, wherein said booster doses are administered biannually.

81) The method of claim 78, wherein said booster doses are administered quarterly.

82) The method of claim 78, wherein said booster doses are administered monthly.

83) A method for monitoring the efficacy of perinatal cell and Treg therapy for preventing type 1 and type 2 diabetes in a patient identified as having antibodies associated with type 1 diabetes, comprising: measuring levels of pancreatic islet cell autoantibodies in the patient; monitoring blood glucose levels in the patient; and assessing insulin production and function in the patient.

84) The method of claim 83, wherein a decrease in pancreatic islet cell autoantibody levels, stabilization of blood glucose levels, and restoration of insulin production and function indicate efficacy of perinatal cell and Treg therapy.

85) The method of claim 83, further comprising imaging pancreatic tissue to assess regeneration and preservation of pancreatic islet cells.

86) The method of claim 83, wherein said monitoring is performed periodically following administration of perinatal cells and Tregs.

87) The method of claim 83, wherein said monitoring is performed using non-invasive imaging techniques.

88) The method of claim 83, wherein said monitoring is performed using biomarker assays.

89) The method of claim 83, wherein said monitoring is performed using clinical assessments.

90) A method for selecting patients for perinatal cell and/or Treg therapy for preventing type 1 and type 2 diabetes, comprising: identifying patients with antibodies associated with type 1 diabetes; assessing pancreatic beta-cell function in said patients; and selecting patients with preserved pancreatic beta-cell function for perinatal cell and Treg therapy.

91) The method of claim 90, wherein said pancreatic beta-cell function is assessed using glucose tolerance tests.

92) The method of claim 90, wherein said pancreatic beta-cell function is assessed using C-peptide levels.

93) The method of claim 90, wherein said pancreatic beta-cell function is assessed using insulin secretion assays.

94) The method of claim 90, wherein said patients are further selected based on age, gender, genetic predisposition, or other clinical parameters.

95) A method for preventing type 1 and type 2 diabetes in a patient identified as having antibodies associated with type 1 diabetes, comprising administering perinatal cells, Tregs, and a pharmaceutical composition comprising an immunomodulatory agent, a growth factor, or an encapsulating material to said patient.

96) The method of claim 95, wherein said perinatal cells, Tregs, and pharmaceutical composition are administered via different routes of administration.

97) The method of claim 95, wherein said immunomodulatory agent, growth factor, or encapsulating material is administered before, simultaneously with, or after the administration of perinatal cells and Tregs.

98) The method of claim 95, wherein said immunomodulatory agent is administered periodically to prevent rejection of said perinatal cells and Tregs.

99) The method of claim 95, wherein said growth factor is administered periodically to enhance the survival and function of said perinatal cells and Tregs.

100) The method of claim 95, wherein said perinatal cells, Tregs, and pharmaceutical composition are administered in a sequential manner to maximize therapeutic efficacy.

These aspects cover various aspects of preventing both autoimmune type 1 and type 2 diabetes in patients with associated antibodies using perinatal cell therapy and T regulatory cell therapy, including methods, compositions, kits, monitoring techniques, and patient selection criteria.

### BRIEF DESCRIPTION OF THE DRAWINGS

FIG. 1 shows a photo of intraarterial delivery of Perinatal MSC cells into the pancreas.

### DETAILED DESCRIPTION OF THE INVENTION

The invention, in some embodiments, teaches the application of immunomodulation and regeneration in patients with autoantibodies before the become insulin dependent diabetics. It is known that the immune system allows for recognition and elimination of pathological threats, while selectively ignoring antigens that belong to the body. Traditionally, autoimmune conditions or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. However, in a patient who progress to diabetes immunosuppressants lead to progression of disease and lead to end organ damage. Therefore the goal is to treat the immune system before lead to long term damage, as in this case destruction of the functional capacity of the pancreas to produce insulin and muscle tissue to have functional insulin receptors.

It should be noted that, as used in this specification and the appended claims, the singular forms “a,” and, “the” include plural referents unless the context clearly dictates otherwise. Thus, for example, reference to “a cell” includes one or more of such cells and reference to “the flask” includes reference to one or more of such flasks.

As used herein, the term “isolated cell” refers to a cell that has been isolated from a tissue, including from other cells of that tissue.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

As used herein, the definition of the term “stem cells or perinatal cells” relates to a cell capable of making copies of itself and having the capacity to differentiate into other types of cells. In some situations, “stem cells” is associated with research that may help patients suffering from previously incurable diseases. In other situations, “stem cells” represent “repair cells” of the body.

This same principle applies to ranges reciting only one numerical value as a minimum or a maximum. Furthermore, such an interpretation should apply regardless of the breadth of the range or the characteristics being described.

An initial overview of embodiments is provided below, and specific embodiments are then described in further detail. This initial summary is intended to aid readers in understanding the disclosure more quickly and is not intended to identify key or essential technological features, nor is it intended to limit the scope of the example subject matter.

Disclosed are perinatally tissue derived immunomodulatory cells and methods of extraction, expansion and potentiation therapeutic activity of said cells. In some cases, submembrane derived cells from perinatal tissue are isolated and expanded under physiological or disease-associated conditions ex vivo in order to retain or enhance therapeutic properties prior to administration for treatment of degenerative and/or autoimmune conditions.

To those of skill in the art, it has become known that there exist numerous types of stem cells, there are hematopoietic stem cells, which are responsible for production of blood. There are embryonic stem cells, which are capable of generating any tissue in the body, however, suffer from the predisposition to generate tumors termed “teratomas”. There are tissue-specific stem cells, such as brain stem cells in the dentate gyrus and subventricular zone, whose proliferation is associated with higher mental activity and resistance to depression. In one specific example, a stem/perinatal cell can be a mesenchymal stem cell (MSC).

While other reasoning is contemplated, MSCs can be useful due to a) MSCs are true “repair cells”. In many conditions, healing is associated with MSCs producing growth factors, which coordinate numerous cell types to initiate and maintain recovery of bodily function after injury; b) MSCs do not require matching between donor and recipient. This can be important because it allows for the use of the stem cell as a therapeutic, similar to an active agent or drug. A such, a large quantity of cells can be grown, standardized, characterized and subsequently used to treat a variety of different patients; c) MSCs act as “biological anti-inflammatoires”. Specifically, not only do MSC promote healing of injured tissues, but they also reduce inflammation. One very important aspect of MSC reducing inflammation is that they only reduce inflammation in the presence of tissue injury. This means that if MSCs are injected into a patient with no inflammation, then the MSCs do not produce anti-inflammatory products. In other cases, when a patient suffers from an inflammatory condition, the MSCs actually produce more anti-inflammatory agents in order to reduce the inflammation. Essentially, the MSC act as a “natural” anti-inflammatory cell, specifically “knowing” how many and what factors to producing; d) MSCs home to tissues of injury. It is known that most injury is associated with generation of blood clotting. This process results in lack of oxygen to the damaged tissue. When tissue lacks oxygen, cells of the tissue start producing “chemokines” which act as local beacons, calling in MSCs only to the area of tissue injury.

In addition to conceptual advantages of MSC, one useful aspect of this particular family of stem cells is that they have been used in numerous clinical trials with an excellent safety profile. While not all clinical trials have shown specific improvements from an efficacy perspective, adverse reactions to MSC administration, whether intravenous, intrathecal, intra-arterial, intramuscular, or the like, have largely not been observed.

Unfortunately, there appears to be room for improvement in the area of stem cells in general, and particularly in the area of mesenchymal stem cells. We believe that types of mesenchymal stem cells are needed because of: a) need for enhanced growth factor production by MSC; b) need for augmentation of homing by MSC to tissue of injury; and c) desire for MSC to be more economical. Here we describe the previous type of MSC from perinatal tissue, which have been modified to possess therapeutic activity that is superior to other stem cell types.

Those skilled in the art recognize numerous types of stem cells, including, for example, hematopoietic stem cells, which are responsible for the production of blood, tissue-specific stem cells, such as brain stem cells in the dentate gyrus and subventricular zone, whose proliferation is associated with higher mental activity and resistance to depression. Another example includes embryonic stem cells, which are capable of generating any tissue in the body; however, they suffer from the predisposition to generate tumors termed “teratomas.”

While numerous types of stem cells have been discovered, and most likely, many will be discovered, one type of useful class of stem cells are mesenchymal stem cells (MSCs). One reason for using MSCs as compared to other cell types for development of therapeutics is due to the fact that MSCs are true “repair cells.” In many conditions, healing is associated with MSCs producing growth factors, which coordinate numerous cell types to initiate and maintain recovery of bodily function after injury. Another reason is due to the fact that MSC's do not require matching between donor and recipient. This is important because it allows for the use of the stem cell as an “active agent,” such as a “drug” or other therapeutic agent. As such, large quantities MSCs can be grown, standardized, characterized, and subsequently used to treat a variety of different patients. In yet another reason, MSCs can act as “biological anti-inflammatories.” Specifically, not only do MSCs promote healing of injured tissues, but they also reduce inflammation. One important aspect of MSC reducing inflammation is that they only reduce inflammation in the presence of tissue injury. This means that if MSCs are injected into a patient with no inflammation, then the MSCs do not produce anti-inflammatory products. In other cases, when the patient suffers from an inflammatory condition, the MSCs actually produce more anti-inflammatory agents in order to reduce the inflammation. Essentially, the MSCs act as “natural” anti-inflammatory cells, specifically “knowing” how many and what factors to produce. In a further example, MSCs can home in on injured tissues. It is known that most injury is associated with generation of blood clotting. This process results in lack of oxygen to the damaged tissue. When tissue lacks oxygen, cells of the tissue start producing “chemokines” which act as local beacons, calling in MSC only to the area of tissue injury.

In addition to conceptual advantages of MSC, one very important aspect of this particular family of stem cells is that they have been used in numerous clinical trials with an excellent safety profile. While not all clinical trials have shown specific improvements from an efficacy perspective, adverse reactions to MSC administration, whether intravenous, intrathecal, intra-arterial, or intramuscular, have largely not been observed.

The present disclosure provides use of novel stem cell types which includes techniques for deriving stem cells possessing immunomodulatory, immunomodulatory, anti-inflammatory, and increasing muscle sensitivity from perinatal tissue. Perinatal tissue can include tissues such as such as the placenta, umbilical cord, cord blood, amniotic fluid, and the like. In some examples, the manipulation of stem cell “potency” can be achieved through hypoxic manipulation, growth under non-xenogeneic conditions, as well as addition of epigenetic modulators.

The presently disclosed perinatal cells can be cultured under hypoxia, in one example, in order to induce and/or augment expression of chemokine receptors. One specific example of such a receptor is CXCR-4. One example population of stem cells includes perinatal tissue mesenchymal cells. A variety of techniques can be utilized to extract the isolated stem cells, and any such technique that allows such extraction without significant damage to the stem cells is considered to be within the present scope. In one example, a method of culturing stem cells from the mammalian perinatal tissue can include the dissection of perinatal tissue. In one specific example, perinatal tissue can be collected and washed to remove blood, Wharton's Jelly, and any other material associated with the submembrane layer. In yet another non-limiting example the cord tissue can be washed multiple times in a solution of Phosphate-Buffered Saline (PBS) such as Dulbecco's Phosphate-Buffered Saline (DPBS). In some examples, the PBS can include a platelet lysate (i.e. 10% PRP lysate of platelet lysate-liquid or lyophilized). Any remaining undesired portion of the perinatal tissue can then be removed and discarded. The remaining perinatal tissue can then be placed interior side down on a substrate with the tissue of interest being in contact with the substrate. Any size of perinatal tissue section can be placed interior side down on a substrate. In one example, the perinatal tissue section can be the same or substantially the same size as the substrate. In another example, the dissected perinatal tissue can be cut into smaller sections (e.g. 1-3 mm) which can be placed directly onto the substrate.

A variety of substrates are contemplated upon which the submembrane layer (SL) can be placed. In one aspect, for example, the substrate can be a polymeric material, such as, for example, a solid polymeric material. One example of a solid polymeric material can include a cell culture dish. The cell culture dish can be made of a cell culture treated plastic as is known in the art. In one specific example, the SL can be placed upon the substrate of the cell culture dish without any additional pretreatment to the cell culture treated plastic. In another example, the substrate can be a semi-solid cell culture substrate. Such a substrate can include, for example, a semi-solid culture medium including an agar or other gelatinous base material.

Following placement of the submembrane layer on the substrate, the submembrane layer is cultured in a suitable medium. In some examples, the culture media can be free of animal and human components or contaminants. The culture can then be cultured under either normoxic or hypoxic culture conditions for a period of time sufficient to establish primary cell cultures. (e.g. 3-7 days in some cases). After primary cell cultures have been established, the submembrane layer tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1×glutamine, 1×. NEAA, and 0.1-20% PRP lysate or platelet lysate. Another example of suitable media can include a base medium of DMEM low glucose without phenol red, 1×. glutamine, 1×. NEAA, with or without 1000 units of heparin and 20% PRP lysate liquid or lyophilized platelet lysate. In another example, cells can be cultured directly onto a semi-solid substrate of DMEM low glucose without phenol red, 1×glutamine, 1×. NEAA, and 20% PRP lysate liquid or lyophilized platelet lysate. In a further example, culture media can include a low glucose medium (500-1000 mg/ml) containing 1×. Glutamine, 1×. NEAA, 1000 units of heparin. In some aspects, the glucose can be 1000-4000 mg/mL, and in other aspects the glucose can be high glucose at 4000-6000 mg/mL. These media can also include 0.1%-20% PRP lysate liquid or lyophilized platelet lysate. In yet a further example, the culture medium can be a semi-solid with the substitution of acid-citrate-dextrose ACD in place of heparin, and containing low glucose medium (500-1000 mg/ml), intermediate glucose medium (1000-4000 mg/mL) or high glucose medium (4000-6000 mg/mL), and further containing 1×. Glutamine, 1×. NEAA, and 0.1%-20% PRP lysate liquid or lyophilized platelet lysate. In some aspects, the cells can be derived, subcultured, and/or passaged using TrypLE. In another aspect, the cells can be derived, subcultured, and/or passaged without the use of TrypLE or any other enzyme that is xeno-free.

In other embodiments of the invention, purified populations of immunomodulatory cells can be obtained from human perinatal tissue. As used herein, “purified” means that at least 90% (e.g., 91, 92, 93, 94, 95, 96, 97, 98, or 99%) of the cells within the population are immunomodulatory cells. As used herein, “immunomodulatory cells” refers to mammalian cell. Within the context of the current invention immunomodulatory cells can be isolated from perinatal tissues obtained with informed consent. Typically, after a perinatal tissue is obtained in a hospital or clinic, the tissue is placed in a hypothermic preservation solution, such as FRS solution from Biolife Solutions (catalog #HTS-FRS) and stored at 4° C. To begin isolating perinatal tissue derived cells, the hypothermic preservation solution can be removed by washing in a buffer, such as Hank's basic salt solution, that is free of Mg2+, Ca2+, and phenol free. The perinatal tissue can be cut into cross sections in the presence of a buffer, and then the cross-sections can be cut longitudinally into two pieces while avoiding any venous or arterial tissue. If any blood is released into the buffer while cutting the cord, the contaminated buffer is replaced with fresh buffer. The longitudinal pieces of cord can be dissected to remove venous and arterial tissue such that the resulting cord lining (i.e., the gelatinous cord material) is substantially free of venous and arterial tissue. As used herein “substantially free of venous and arterial tissue” indicates that as much visible venous and arterial tissue has been removed as possible with manual dissection. The immunomodulatory cells can be obtained from the dissected submembrane lining by culturing the longitudinal pieces of submembrane lining on a fibronectin coated solid substrate (e.g., a plastic culture device such as a chambered slide or culture flask). The gelatinous surface of the cord lining can be placed in contact with the fibronectin coated solid substrate while the upper surface (i.e., the surface not in contact with the fibronectin coated solid substrate) can be covered with a solid substrate such as a coverslip. Low glucose (i.e., .ltoreq.1 g/L glucose) growth medium can be added and the culture device incubated for a time sufficient for cells to migrate from the cord lining to the fibronectin coated solid substrate (e.g., 7 to 10 days). Unless otherwise indicated, cells are cultured at 37° C. in a standard atmosphere that includes 5% CO2. Relative humidity is maintained at about 100%. After have adhered to the surface of the fibronectin coated solid substrate, the coverslip can be removed, and the adhered cells can be washed in a buffer such as phosphate-buffered saline (PBS). A growth medium that can be used for culturing Immunomodulatory cells is low glucose Dulbecco's Modified Essential Media (DMEM) containing vitamins (choline chloride, D-Calcium pantothenate, Folic Acid, Nicotinamide, Pyridoxal hydrochloride, Riboflavin, Thiamine hydrochloride, and i-Inositol), and non-essential amino acids (glycine, L-alanine, L-Asparagine, L-Aspartic acid, L-Glutamic Acid, L-Proline, and L-Serine). Low glucose DMEM can be supplemented with 10% to 20% serum (e.g., fetal bovine serum (FBS) or human serum), one or more antibiotics (e.g., gentamycin, penicillin, or streptomycin), and glutamine or a stabilized dipeptide of L-alanyl-L-glutamine (e.g., GlutaMax from Invitrogen). In one embodiment, a growth medium can include low glucose DMEM containing vitamins and non-essential amino acids, 15% FBS, 1 to 3% antibiotic (e.g., 2% or 2×. gentamycin), and 0.7 to 1.5% (e.g., 1%) of glutamine or a stabilized dipeptide of L-alanyl-L-glutamine. Such a growth medium can be further supplemented with 1 to 100 ng/ml of a growth factor (e.g., basic fibroblast growth factor (bFGF), leukemia inhibitory factor (LIF), or epidermal growth factor (EGF).

In some embodiments, a growth medium further includes insulin, transferrin, selenium, and sodium pyruvate. A particularly useful growth medium can include low glucose DMEM containing vitamins and non-essential amino acids, 15% serum, 1 to 3% antibiotic (e.g., 2% or 2×. gentamycin), 0.7 to 1.5% of glutamine or a stabilized dipeptide of L-alanyl-L-glutamine (e.g., 1% or 1×. GlutaMax), 1 to 100 ng/ml of a growth factor (e.g., 10 ng/ml bFGF and 10 ng/ml LIF), 0.1 mg/ml to 100 mg/mL of insulin (10 mg/mL), 0.1 mg/mL to 100 mg/ml of transferrin (e.g., 0.55 mg/ml transferring), 0.1 μg/ml to 100 μg/ml selenium (e.g., 0.5 μg/mL selenium), and 0.5 to 1.5% sodium pyruvate (e.g., 1% sodium pyruvate). In some embodiments, such a growth medium further includes 0.05 μg/mL to 100 μg/mL of putrescine (e.g., 10 μg/mL putrescine) and 10 ng/ml of EGF. For embodiments in which an animal free medium is desired, human serum (e.g., 15% human serum) can be used in place of fetal bovine serum or PRP lysate liquid or lyophilized platelet lysate.

In some embodiments of the invention, it is necessary to subculture immunomodulatory cells, TrypZean (Sigma Chemical Co.) can be used to release cells from the solid substrate. The resulting cell suspension can be pelleted and washed with PBS, then seeded into cell culture flasks at approximately 1000 cells/cm2 in a growth medium. Clonal lines of immunomodulatory cells can be established by plating the cells at a high dilution and using cloning rings (e.g., from Sigma) to isolate single colonies originating from a single cell. Cells are obtained from within the cloning ring using trypsin then re-plated in one well of a multi-well plate (e.g., a 6-well plate). After cells reach >60% confluency (e.g., >70% confluency), the cells can be transferred to a larger culture flask for further expansion. Immunomodulatory cells can be assessed for viability, proliferation potential, and longevity using techniques known in the art. For example, viability can be assessed using trypan blue exclusion assays, fluorescein diacetate uptake assays, or propidium iodide uptake assays. Proliferation can be assessed using thymidine uptake assays or MTT cell proliferation assays. Longevity can be assessed by determining the maximum number of population doublings of an extended culture.

Immunomodulatory cells can be immunophenotypically characterized using known techniques. For example, the cells can be fixed (e.g., in paraformaldehyde), permeabilized, and reactive sites blocked (e.g., with serum albumin), then incubated with an antibody having binding affinity for a cell surface antigen. The antibody can be detectably labeled (e.g., fluorescently or enzymatically) or can be detected using a secondary antibody that is detectably labeled. In some embodiments, the cell surface antigens on Immunomodulatory cells can be characterized using flow cytometry and fluorescently labeled antibodies.

Immunomodulatory cells also can be characterized based on the expression of one or more genes. Methods for detecting gene expression can include, for example, measuring levels of the mRNA or protein of interest (e.g., by Northern blotting, reverse-transcriptase (RT)-PCR, microarray analysis, Western blotting, ELISA, or immunohistochemical staining)

Immunomodulatory cells can be cryopreserved by suspending the cells (e.g., up to 5×106 cells/mL) in a cryopreservative such as dimethylsulfoxide (DMSO, typically 10%). In some embodiments, a freezing medium such as CryoStor from Biolife solutions is used to cryopreserve the cells. After adding cryopreservative, the cells can be frozen (e.g., to −90° C.). In some embodiments, the cells are frozen at a controlled rate (e.g., controlled electronically or by suspending the cells in a bath of 70% ethanol and placed in the vapor phase of a liquid nitrogen storage tank. When the cells are chilled to −90° C., they can be placed in the liquid phase of the liquid nitrogen storage tank for long term storage. Cryopreservation can allow for long-term storage of these cells for therapeutic use.

These cells isolated according to the above methodology may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition, or alternatively, <1%, <2%, <3%, <4%, <5%, <6%, <7%, <8%, <9%, or <10% of the population of cells may express CD14 and/or CD45. The umbilical cord cells of the invention may further possess markers selected from STRO-1, CD105, CD54, CD56, CD106, HLA-I markers, vimentin, ASMA, collagen-1, fibronectin, LFA-3, ICAM-1, PECAM-1, E-selectin, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In particular embodiments, cells of the invention lack expression of CD90, CD105 and CD34 but possess CD56, and/or CD73 expression.

In some embodiments said immunomodulatory cells of the invention are admixed with T regulatory cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the placental body, placenta, umbilical cord tissue with interleukin-10, and/or interleukin 35 expression. In some embodiments, the population of cells, the population of perinatal tissue cells, or the population of endothelial cells express hTERT and Oct-4 but does not express a STRO-1 marker.

In some embodiments, the population of cells, the population of perinatal tissue cells has an ability to undergo cell division in less than 36 hours in a growth medium. In some embodiments, the population of cells, the population of perinatal tissue cells has an ability to proliferate at a rate of 0.9-1.2 doublings per 36 hours in growth media. In some embodiments, the population of cells, the population of perinatal tissue cells has an ability to proliferate at a rate of 0.9, 1.0, 1.1, or 1.2 doublings per 36 hours in growth media. The population of cells, population of perinatal tissue cells may produce exosomes capable of inducing more than 50% proliferation when the exosomes are cultured with human perinatal tissue endothelial cells. The induction of proliferation may occur when the exosomes are cultured with the human perinatal tissue endothelial cells at a concentration of 10, 20, 30, 40, 50, 60, 70, 80, 90, 100, 110, 120, 130, 140, 150, 160, 170, 180, 190, 200, or more exosomes per cell. Exosomes produced by cells described herein, in some embodiments express CD9, CD63 and/or CD81.

In some embodiments, a population of cells, including a population of perinatal tissue derived cells alone, are administered to an individual, including an individual having and acute or chronic pathology, wherein the population of cells may be administered via any suitable route, including as non-limiting examples, intramuscularly and/or intravenously.

In some embodiments, a population of perinatal tissue derived cells is optionally obtained, the population is then optionally contacted via culturing with a population of progenitor for T regulatory cells, wherein the culturing conditions allow for the generation of T regulatory cells, then the generated T regulatory cells are administered to an individual.

In some embodiments of the invention, administration of cells of the invention is performed for suppression of an inflammatory and/or autoimmune disease. In these situations, it may be necessary to utilize an immune suppressive/or therapeutic adjuvant. Immune agents are known in the art and can be selected from a group comprising of: teplizumab, cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric. acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

The optimal dose of perinatal tissue MSCs for some embodiments will be in the range of doses used for autologous, mononuclear bone marrow transplantation. For fairly pure preparations of perinatal tissue MSC, optimal doses in various embodiments will range from 104 to 108 perinatal tissue MSCs cells/kg of recipient mass per administration. In some embodiments, the optimal dose per administration will be between 105 to 107 perinatal tissue MSCs cells/kg. In many embodiments, the optimal dose per administration will be 5×105 to 5×106 perinatal tissue MSCs cells/kg. By way of reference, higher doses in the foregoing are analogous to the doses of nucleated cells used in autologous mononuclear bone marrow transplantation. Similarly, perinatal MSCs have been administered in doses of 100M to 400M in patients with no toxicity issues.

It is to be appreciated that a single dose may be delivered all at once, fractionally, or continuously over a period of time. The entire dose also may be delivered to a single location or spread fractionally over several locations. It may deliver intraarterially, intravenously or in combination.

In various embodiments, perinatal tissue MSCs may be administered in an initial dose, and thereafter maintained by further administration of perinatal tissue MSCs. Perinatal tissue MSCs may be administered by one method initially, and thereafter administered by the same method or one or more different methods. The subject's perinatal tissue MSC levels can be maintained by the ongoing administration of the cells. Various embodiments administer the perinatal tissue MSCs either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals, but treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regiments can be ascertained by the skilled artisan, from this disclosure, the documents cited herein, and the knowledge in the art. The dose, frequency, and duration of treatment will depend on many factors, including the nature of the disease, the subject, and other therapies that may be administered. Accordingly, a wide variety of regimens may be used to administer perinatal tissue MSCs.

Perinatal tissue MSCs may be administered in many frequencies over a wide range of times. In some embodiments, perinatal tissue MSCs are administered over a period of less than one day. In other embodiments, they are administered over two, three, four, five, or six days. In some embodiments, perinatal tissue MSCs are administered one or more times per week, over a period of weeks. In other embodiments they are administered over a period of weeks for one to several months. In various embodiments, they may be administered over a period of months. In others they may be administered over a period of one or more years. Generally, lengths of treatment will be proportional to the length of the disease process, the effectiveness of the therapies being applied, and the condition and response of the subject being treated.

The MSCs administered in an environment that is already protolerogenic enhances said tolerogenicity, however for the practice of the invention, the augmentation of tolerance is supported by the administration of augmented T regulatory cells, which provide not only direct angiogenic support, but also act as tolerogenic and immunomodulatory cells that decrease rejection. For the practice of the invention, we describe below different ways in which MSCs modulate the immune system such that practitioners of the invention may utilize known means in the art. In another embodiment of the invention, the protection of the pancreatic islets (cells/tissues/organs) are facilitated by infusion of augmented T regulatory cells. The T regulatory cells can be administered before perinatal MSCs induce a tolerogenic process. In another embodiment, they are administered after the perinatal MSCs are transplanted. This process may involve one or more doses of the augmented T regulatory cells.

The invention, in some embodiments, teaches the utilization of T regulatory cells, whereby said cells possess immune regulatory and repair properties. It is known that a cardinal feature of the immune system allows for recognition and elimination of pathological threats, while selectively ignoring antigens that belong to the body. Traditionally, autoimmune conditions or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia.

The current approach described teaches use of T regulatory cells which possess immunological and regenerative properties.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition, or alternatively, <1%, <2%, <3%, <4%, <5%, <6%, <7%, <8%, <9%, or <10% of the population of cells may express CD14 and/or CD45. The perinatal cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof.

The T regulatory cells which are regenerative are derived by obtaining a peripheral blood mononuclear cells via an apheresis from a patient. The fresh or frozen leuko-pack is enriched, depleted and separated for a group of factors such as FOXP3, CD4, CD8, CD25, and/or CD39 using antibody bead based and density gradient optimization. The augmentation of the T cells is performed via either directed co-culture of the perinatal tissue MSCs which have been stimulated with one of many factors such as gamma interferon. The supernatant of the stimulated perinatal tissue MSCs and/or the stimulated perinatal cells are co-cultured with the T cells to produce the augmented T regulatory cells which have better immunomodulatory and anti-rejection properties compared to standard T cells. The cells may also be derived from IPS cells to enable larger scalability. These augmented T regulatory cells are then administered to the patient before, during, and/or after the perinatal MSC transplantation in doses to 10M to 1B cells IV. This will allow for repeat dosing to best optimize the patient's immunomodulatory regimen. In some cases, the patient may receive teplizumab or rhGAD65 before receiving the perinatal MSC or T regulatory cell therapy as to augment beta islet cell function in a synergistic manner or in the case of lack of efficacy. A dedicated electronic glucose monitoring system was created using all three IOS, android and standard web-based data input and capture system which will notify the patient and the clinical care team that the patients glucose level is out of range. This is a key component of the program to optimize compliance, safety and effective clinical management of the patient and minimize complications such as diabetic ketoacidosis, hospital admissions and possible death.

Experimental Procedure: Intraarterial Delivery of Perinatal Cells in Patients with Type 1 Diabetes Antibodies

Objective: The objective of this experimental procedure is to assess the safety and efficacy of delivering perinatal cells via intraarterial injection to patients with type 1 diabetes autoimmune antibodies who do not require insulin, Assessments will be based on the change in beta islet function as assessed by C-peptide levels, HgA1C, glucose tolerance test, along with autoantibodies.

### Materials Required:

Perinatal cells (obtained from Human GMP Cell Bank)
Catheters for intra-arterial delivery
Heparin
Sterile saline solution
Intravenous catheters
Blood collection tubes
Glucose solution for oral glucose tolerance test (OGTT)
Laboratory equipment for C-peptide level analysis
Laboratory equipment for autoantibody analysis

### Experimental Procedure:

### Patient Selection:

Patients diagnosed with who have detectable autoantibodies against pancreatic islet cells who do not require insulin and are stable medical therapy for 30 days.

Ensured patients meet inclusion criteria such as age, health status, and willingness to participate in the study.

Obtained informed consent from each patient after explaining the nature and potential risks and benefits of the procedure.

### Preparation of Perinatal Cells:

Perinatal cells from Human GMP Cell Bank used in prior studies with adequate safety history were be utilized
Processed the perinatal cells according to established protocols to obtain a suspension suitable for injection.

### Intraarterial Injection:

Prepared the patient for the procedure in a sterile operating room or interventional radiology suite.

Administered local anesthesia at the site of arterial access (common femoral artery or radial artery).

Patient given adequate heparin to obtain ACT>200. Based on clinician skill and preference—not always required.

Inserted a catheter with guidewire into the artery under fluoroscopic or ultrasound guidance.

Injected 100M perinatal cells suspended in sterile saline solution directly into the artery at 10M cells per minute.

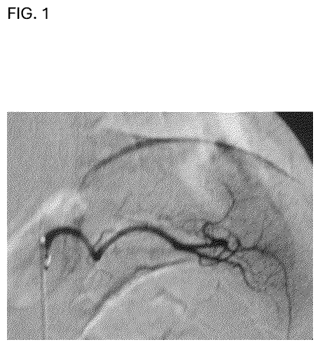

Monitored the patient closely for any signs of adverse reactions during and after the injection-allergic reactions, hematoma, infection, injury to artery or pancreas, stroke, death. FIG. 1 shows a photo of intraarterial delivery of Perinatal MSC cells into the pancreas.

### Post-Injection Monitoring:

Monitored vital signs, including blood pressure, heart rate, and oxygen saturation, continuously during the procedure and post-injection period.

Performed regular blood draws to assess C-peptide levels and autoantibody titers before and after the injection at specified intervals.

### Assessment of Beta Islet Function:

Performed OGTT before and after the injection to evaluate glucose tolerance and insulin response.

Measured C-peptide levels in the blood samples collected to assess beta islet function.

Compared pre- and post-injection C-peptide levels and OGTT results to determine improvement in beta islet function.

### Evaluation of Autoantibodies:

Analyzed blood samples collected before and after the injection to quantify autoantibody levels.

Determined the percentage reduction in autoantibody titers following the intraarterial delivery of perinatal cells.

### Data Analysis:

Compiled and analyzed the data obtained from C-peptide measurements, OGTT results, and autoantibody assays.

Calculated the percentage improvement in beta islet function and reduction in autoantibody titers compared to baseline levels.

Performed statistical analysis to determine the significance of the observed changes.

### Follow-Up:

Scheduled follow-up appointments with patients to monitor their clinical status and any potential adverse effects.

Repeated assessments of beta islet function and autoantibody levels at regular intervals to evaluate the long-term effects of the treatment.

### Documentation:

Maintained detailed records of each patient's medical history, procedure details, and post-treatment outcomes.

Documented any adverse events or complications encountered during the study.

### Conclusion:

The experimental procedure outlined above aimed to evaluate the efficacy of intraarterial delivery of perinatal cells in patients with type 1 diabetes antibodies. By assessing changes in beta islet function, clinical and other laboratory parameters, the study provided valuable insights into the therapeutic benefits of this novel perinatal cell therapy approach.

### Summary of Clinical Data

Three patients successfully consented, enrolled and treated. There were no severe adverse events over during the infusion and following up period. The only adverse event related to product or procedure during the infusion was a small bruise at the infusion catheter introduction site with no hematoma that resolved in 7 days. Results are shown in Table 1.

TABLE 1

| Parameter | Patient | Baseline | 1 month | 3 months | 6 months | 12 months |
|---|---|---|---|---|---|---|
| HgA1c | 1 | 6.2 | 6.4 | 6.0 | 5.7 | 5.6 |
| (mmol/mol) | 2 | 5.9 | 6.1 | 5.4 | 5.1 | 5.2 |
| | 3 | 6.5 | 6.4 | 5.9 | 5.5 | 5.3 |
| Insulin(IU/kg/day) | 1 | 0 | 0 | 0 | 0 | 0 |
| | 2 | 0 | 0 | 0 | 0 | 0 |
| | 3 | 0 | 0 | 0 | 0 | 0 |
| Fasting C-peptide | 1 | 0.94 | 1.00 | 1.53 | 1.52 | 1.71 |
| (nmol/L) | 2 | 1.1 | 1.20 | 1.76 | 2.11 | 2.34 |
| | 3 | 0.99 | 1.02 | 1.69 | 1.88 | 2.13 |

1. A method for preventing type 2 autoimmune diabetes in patients with one or more autoantibodies targeting islet cell antibodies (ICA), comprising administering perinatal cell therapy.

2. The method of claim 1, wherein the perinatal cell therapy comprises mesenchymal stem cells derived from perinatal tissue.

3. The method of claim 1, wherein the perinatal cell therapy comprises umbilical cord blood-derived stem cells.

4. The method of claim 1, wherein the perinatal cell therapy comprises placental-derived stem cells.

5. The method of claim 1, wherein the perinatal cell therapy is administered via intramuscular injection.

6. The method of claim 1, wherein the perinatal cell therapy is administered via intravascular injection.

7. The method of claim 1, wherein the perinatal cell therapy is administered via intra-pancreatic injection.

8. The method of claim 1, further comprising administering a carrier solution with the perinatal cell therapy.

9. The method of claim 8, wherein the carrier solution comprises a saline solution or a biocompatible hydrogel.

10. The method of claim 1, further comprising administering immunosuppressive agents and/or cytokines before the perinatal cell therapy.

11. The method of claim 1, further comprising administering anti-inflammatory agents along with the perinatal cell therapy.

12. The method of claim 1, further comprising administering growth factors before or along with the perinatal cell therapy.

13. The method of claim 1, wherein the efficacy of the perinatal cell therapy is assessed by monitoring levels of HbA1c, c-peptide, autoantibodies and/or Clark score.

14. The method of claim 1, wherein the efficacy of the perinatal cell therapy is assessed by monitoring frailty and sarcopenia.

15. A pharmaceutical composition for preventing type 2 autoimmune diabetes, comprising perinatal cells and a carrier solution.

16. The pharmaceutical composition of claim 15, wherein the perinatal cells are selected from mesenchymal stem cells, hematopoietic stem cells, amniotic fluid-derived stem cells, umbilical cord blood-derived stem cells, and placental-derived stem cells.

17. (canceled)

18. A method for preventing type 2 autoimmune diabetes in a patient at risk thereof, comprising administering perinatal cell therapy in combination with pancreatic islet transplantation to restore insulin production and glucose regulation.

19. The method of claim 18, wherein the pancreatic islet transplantation is performed using allogeneic or xenogeneic islets.

20. The method of claim 1, further comprising administering gene therapy to modulate immune responses and promote tolerance to pancreatic antigens.
